(19) United States
(12) Patent Application Publication
(10) Pub. No.: US 2014/0316272 A1
(43) Pub. Date: Oct. 23, 2014

(54) ULTRASOUND DIAGNOSIS APPARATUS

(71) Applicants: Kabushiki Kaisha Toshiba, Minato-ku (JP); Toshiba Medical Systems Corporation, Otawara-Shi (JP)

(72) Inventors: Hideki KOSAKU, Nasushiobara-Shi (JP); Hiroyuki Shikata, Nasushiobara-Shi (JP); Yasuhiro Ona, Nasushiobara-Shi (JP); Koichi Shibamoto, Nasushiobara-Shi (JP); Kentaro Tsuzuki, Nasushiobara-Shi (JP); Takashi Kubota, Otawara-Shi (JP)

(73) Assignees: Kabushiki Kaisha Toshiba, Minato-ku (JP); Toshiba Medical Systems Corporation, Otawara-Shi (JP)

(21) Appl. No.: 14/322,414

(22) Filed: Jul. 2, 2014

Related U.S. Application Data
(63) Continuation of application No. PCT/JP2013/078882, filed on Oct. 24, 2013.

(30) Foreign Application Priority Data
Oct. 26, 2012 (JP) 2012-236666

(51) Int. Cl. A61B 17/34; A61B 8/00; A61B 8/08

(52) U.S. Cl. CPC A61B 17/3403; A61B 8/0841; A61B 8/4483; A61B 2017/3413
USPC 600/443

(57) ABSTRACT

The ultrasound diagnosis apparatus is provided capable of preventing visibility of the ultrasound image from being impaired when piercing a puncture needle. The ultrasound diagnosis apparatus according to the embodiments includes an image processor and a display controller. The image processor generates an ultrasound image based on echo signals received by the ultrasound probe. The display controller causes a display unit to display, along with the ultrasound image, a marker which indicates allowable change range for the orientation of the puncture needle defined by the guide mechanism for guiding the puncture needle to the puncture target part.

### CROSS-REFERENCE TO RELATED APPLICATIONS

This application is based upon and claims the benefit of priority from Japanese Patent Applications No. 2012-236666, filed on Oct. 26, 2012; the entire contents of all of which are incorporated herein by reference.

### FIELD

The embodiments of the present invention are related to an ultrasound diagnosis apparatus.

### BACKGROUND

Ultrasound diagnosis apparatuses radiate ultrasound pulses to inside a subject from a piezoelectric transducer housed in an ultrasound probe. The ultrasound diagnosis apparatuses then receive reflected waves generated inside the subject by the piezoelectric transducer to perform various processing. As a result, organism information about inside the subject, such as tomographic images, blood flow information, and the like, can be acquired.

An example of medical treatment using the ultrasound diagnosis apparatus is called as ultrasound paracentesis. According to the ultrasound paracentesis, as referring to an ultrasound diagnosis image for a treatment target part of the subject, a puncture needle, such as an injection needle, or the like, is pierced into the subject by an operator for administration of a drag, suction or discharge of the contents, and the like.

A guide mechanism for guiding the puncture needle to a puncture target part is provided to ultrasound probe parts of some of the ultrasound diagnosis apparatuses used in the above-mentioned ultrasound paracentesis. The operator can pierce the puncture needle stably to the puncture target part via the guide mechanism. In addition, there are various kinds of the guide mechanisms, and for example, the guide mechanism is provided directly to the ultrasound probe. In another example, the guide mechanism is provided indirectly to the ultrasound probe via an attachment.

Further, as an index for the operator when piercing the puncture needle into the subject, a straight path (piercing path) which indicates the piercing direction in accordance with the kind of the guide mechanism conventionally has been displayed so as to be overlapped with the ultrasound image.

However, when the operator pierces the puncture needle while seeing a displacement of a vital tissue, the visibility of the vital tissue may be decreased if the piercing path is displayed overlapped with the ultrasound image.

### BRIEF DESCRIPTION OF THE DRAWINGS

FIG. 1 is a model diagram of ultrasound paracentesis technique according to a first embodiment.

FIG. 2 is a system diagram according to the first embodiment.

FIG. 3 is a side view of an ultrasound probe according to the first embodiment.

FIG. 4 is an indicator diagram of an ultrasound image and a guide mechanism mark according to the first embodiment.

FIG. 5 is a flow diagram according to the first embodiment.

FIG. 6 is a first overview of a piercing limit angle for a puncture needle according to the first embodiment.

FIG. 7 is an indicator diagram of the ultrasound image and a puncture needle piercing range according to the first embodiment.

FIG. 8 is a second overview of the piercing limit angle for the puncture needle according to the first embodiment.

FIG. 9 is a plan view of an ultrasound probe according to a second embodiment.

FIG. 10A is a side view of the ultrasound probe according to the second embodiment.

FIG. 10B is a side view of the ultrasound probe according to the second embodiment.

FIG. 11A is an overview of an ultrasound image and a guide mechanism mark according to the second embodiment.

FIG. 11B is an overview of the ultrasound image and the guide mechanism mark according to the second embodiment.

### DETAILED DESCRIPTION

An ultrasound diagnosis apparatus is provided capable of preventing visibility of the ultrasound image from being impaired when piercing a puncture needle.

In order to solve the above problem, the ultrasound diagnosis apparatus according to the embodiments includes an image processor and a display controller.

The image processor generates an ultrasound image based on echo signals received by the ultrasound probe. The display controller causes a display unit to display, along with the ultrasound image, a marker which indicates allowable change range for the orientation of the puncture needle defined by the guide mechanism for guiding the puncture needle to the puncture target part.

It is described hereinafter the embodiments with reference to figures.

### First Embodiment

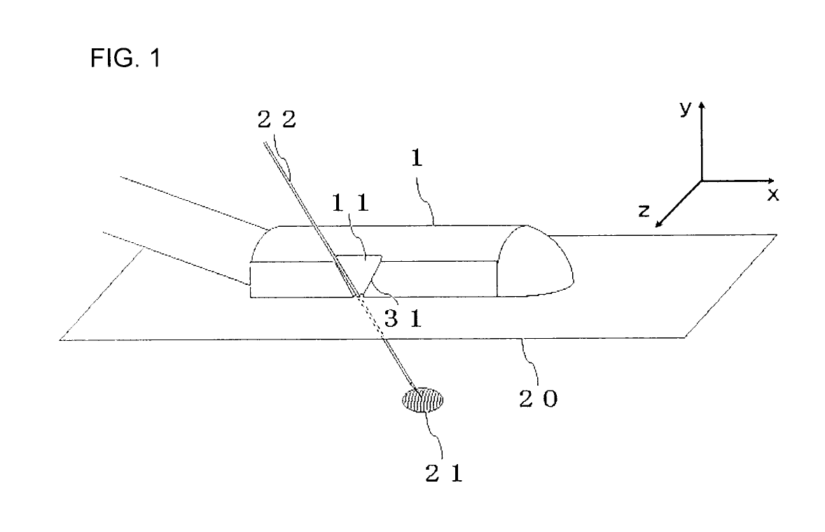

FIG. 1 is a model diagram illustrating ultrasound paracentesis technique. As shown in FIG. 1, an operator presses an ultrasound probe 1 against a subject contact surface 20. Further, the operator pierces a puncture needle 22 into a puncture target part 21 along a wall unit 31 of a guide mechanism 11. In FIG. 1, the guide mechanism 11 has a groove shape, but the shape of the guide mechanism 11 is not limited thereto. The shape may be a hole form, for example.

As the ultrasound probe 1 according to the embodiment, it is described about an intracavitary probe in a body cavity which is inserted into the body cavity to perform ultrasound diagnosis in a surgery using a laparoscope, as an example. However, the embodiment is not limited to the intracavitary probe, and can be applied to other probes.

There is a case where paracentesis, such as RFA (radiofrequency ablation), or the like, is performed during the surgery using the laparoscope. In the surgery, the operator inserts the intracavitary probe (ultrasound probe 1) into inside the abdominal cavity, and further inserts an endoscope from other position. Furthermore, the operator pierces the puncture needle 22 towards the puncture target part 21, such as tumor, in the state where the puncture needle is guided by the guide mechanism 11 which is directly or indirectly provided to the intracavitary probe.

In this case, the operator pierces as referring to the image of the intracavitary probe inserted into the abdominal cavity with the endoscope. Unlike the case where the puncture needle is pierced by the operator being guided by the guide mechanism provided outside the subject, the operator visually recognizes the puncture needle 22 as referring to the image with the endoscope. In this case, the visibility of the puncture needle 22 is therefore easily decreased. For example, it is more difficult for the operator to grasp the piercing direction, or the like, of the puncture needle 22 or the like in referring to the puncture needle 22 via the image of the endoscope, or the like, than in seeing directly the puncture needle 22. In this respect, if a guide range for the puncture needle 22 can be indicated together with the ultrasound image, as in the embodiment, grasping the destination of the puncture needle can be facilitated in the surgery using the laparoscope.

In the RFA, the operator punctures the puncture needle to the target part (center of the tumor, or the like) in the body cavity as observing the ultrasound image. Further, by supplying an electric current to the puncture needle to produce heat around the puncture needle and thus cauterizing the tumor to be necrotized.

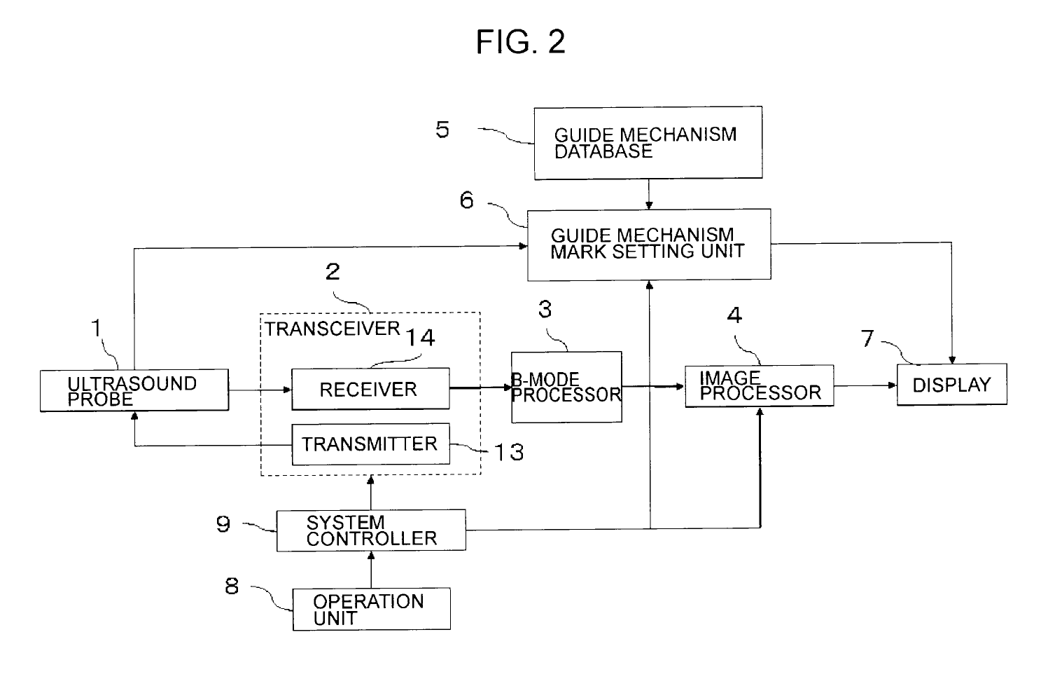

FIG. 2 is a block diagram of the ultrasound diagnosis apparatus according to the first embodiment. The ultrasound diagnosis apparatus includes the ultrasound probe 1, a transceiver 2, a B-mode processor 3, an image processor 4, a guide mechanism database 5, a guide mechanism mark setting unit 6, a display 7, an operation unit 8, and a system controller 9.

The ultrasound probe 1 has the guide mechanism 11 for guiding the puncture needle. Further, the ultrasound probe 1 may be configured on which a not shown attachment provided with the guide mechanism 11 can be mounted.

The ultrasound probe 1 has transducers arranged in plural, a matching layer, and a backing material. The transducers generate ultrasound waves based on the signals (driving pulses) from the transceiver 2. Further, the transducers convert the reflected waves from the subject into electric signals (echo signals). The matching layer matches acoustic impedance between the transducers and the subject. The backing material absorbs the ultrasound waves which are radiated to the opposite side (rear part) of the radiation direction of the ultrasound waves so as to suppress excessive vibration of each transducer.

The transceiver 2 has a transmitter 13 and a receiver 14.

The transmitter 13 repeatedly generates rate pulses for forming transmission ultrasound waves in accordance with instruction from the system controller 9. The transmitter 13 gives each rate pulse a delay time required for determining the directivity of ultrasound beams to generate driving pulses. The transmitter 13 applies each transducer the driving pulses.

The receiver 14 amplifies echo signals from each transducer. The receiver 14 also adds the amplified echo signals from each transducer to create an ultrasound echo signal.

The B-mode processor 3 performs envelope detection on the ultrasound echo signal received from the receiver 14 to generate a B-mode signal corresponding to the amplitude intensity of the ultrasound echo.

The image processor 4 has an image generator, a measurement processor, and a data archive.

The image generator generates a two-dimensional ultrasound image by the B-mode using two-dimensional distribution regarding a predetermined cross-section of the B-mode signal. The image generator also generates a pseudo three-dimensional ultrasound image using three-dimensional distribution related to a predetermined area. Further, the image generator sets a MPR (Multi Planner Reconstruction) position corresponding to a desired reference cross-section using volume data to generate a MPR image corresponding to the MPR position.

The measurement processor measures the inner diameter, volume, and the like, of organs using the generated image and the volume data. The measurement processor also generates image incidental information, such as the inner diameter, volume, and the like, of the organs, based on the measurement result.

The data archive archives the image generated by the image generator and the image incidental information generated by the measurement processor.

The image processor 4 transmits the image or image incidental information to the display 7 in accordance with the instruction from the system controller 9.

The display 7 is caused to display by, for example, a not shown display controller and displays the image or image incidental information transmitted form the image processor 4. Further, the display 7 is controlled by the display controller to display a predetermined operation screen required for operation of the operator.

The operation unit 8 has operation parts (a mouse, a track ball, a key board, and the like) for conducting various instructions from the operator. The operation unit 8 transmits instructions to the transceiver 2 and the image processor 4 via the system controller 9.

In the embodiment, the ultrasound probe 1 and the not shown attachment have IDs for identifying their kind, respectively.

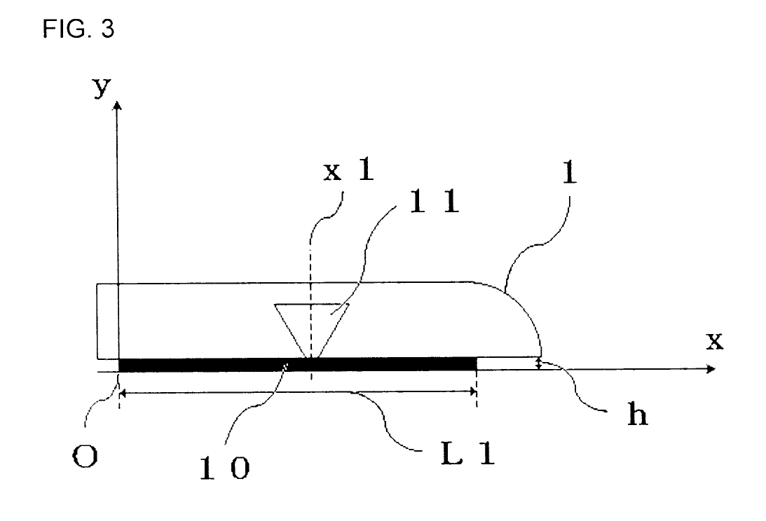

The guide mechanism database 5 stores beforehand shape information and position information, which are uniquely determined with respect to the ID of the guide mechanism 11. The shape information is information representing the shape of the guide mechanism. The position information may be coordinate information representing the position of the guide mechanism 11 in the ultrasound probe 1, as shown in, for example, FIG. 3. Further, when it is the configuration in which an attachment provided with the guide mechanism 11 is attached to the ultrasound probe 1, it may be configured so that the position information is the coordinate information representing the position of the guide mechanism 11 in the attachment. The guide mechanism database 5 corresponds to an example of a “storage”.

With reference to FIG. 3, an example of the coordinate information is described. FIG. 3 is a side view of the ultrasound probe 1. As shown in FIG. 3, the ultrasound probe 1 is provided with an ultrasound wave transceiver 10 having a predetermined length L1. The ultrasound wave transceiver 10 is a part facing the subject contact surface 20 in the ultrasound probe 1 when transmitting and receiving ultrasound waves with the ultrasound probe 1. In addition, in the example of FIG. 3, the x-axis corresponds to the longitudinal direction of the ultrasound wave transceiver 10. Also, the y-axis corresponds to the vertical direction to the subject contact surface 20. Furthermore, in FIG. 3, the origin O corresponds to the end part of the ultrasound wave transceiver 10 in the longitudinal direction. When transmitting and receiving ultrasound waves with the ultrasound probe 1, the x-axis is contacted with the subject contact surface 20 or is positioned along the subject contact surface 20. Further, in FIG. 3, the distance between the x-axis and the lower end of the guide mechanism 11 is set as h. The “lower end” is the end part of the subject contact surface 20 side of the guide mechanism 11 when transmitting and receiving the ultrasound waves with the ultrasound probe 1. Furthermore, the coordinate at the center of the lower end part of the guide mechanism 11 is set as x1.

Such setting of the coordinates is merely an example, and any setting which can present the position of the guide mechanism 11 precisely may be used.

The guide mechanism mark setting unit 6 reads the shape information and position information of the guide mechanism 11 out from the guide mechanism database 5. The guide mechanism mark setting unit 6 creates a guide mechanism mark 18 imitating the shape of the guide mechanism 11, based on the shape information. The guide mechanism mark setting unit 6 transmits the data of the created guide mechanism mark 18 and the position information to the display 7. The embodiment is, however, not limited to the above configuration. For example, if the shape information is the guide mechanism mark 18 itself imitating the shape of the guide mechanism 11, the guide mechanism mark setting unit 6 does not create the guide mechanism mark 18.

The guide mechanism mark setting unit 6 corresponds to an example of the “display controller”. The guide mechanism mark setting unit 6 corresponds to an example of a “marker output unit”, a “selection unit”, or a “marker creator”. The information stored in the guide mechanism database 5 corresponds to an example of “guide mechanism information”. The combination of the guide mechanism mark setting unit 6 with the display 7 corresponds to an example of the “marker output unit”.

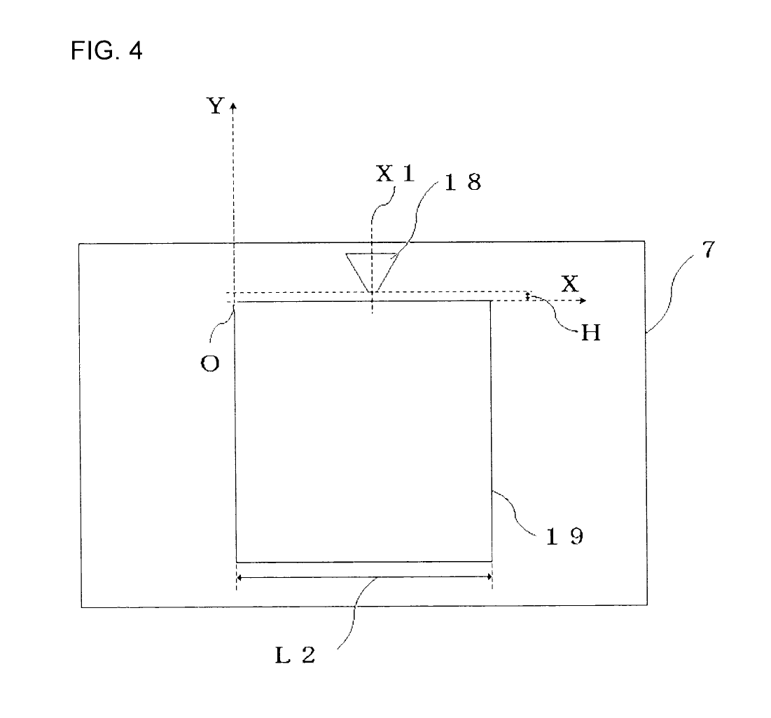

The display 7 displays the guide mechanism mark 18 and an ultrasound image 19, based on the position information. The display may be configured to display controlled by a not shown display controller, for example. The display position for the guide mechanism mark 18 is determined by reflecting the coordinates set in FIG. 3, as shown in, for example, FIG. 4.

FIG. 4 shows the screen representing the arrangement for the guide mechanism mark 18 and the ultrasound image 19 displayed in the display 7. This screen may be configured to be caused to display by a not shown display controller. The origin O in FIG. 3 corresponds to the origin O in FIG. 4. A range L2 for the ultrasound diagnosis shown in FIG. 4 corresponds to the range obtained by enlarging or reducing the length L1 of the ultrasound wave transceiver 10 in the longitudinal direction in FIG. 3 by L2/L1 times. The distance between the boundary line (X-axis) at the upper part of the ultrasound image 19 and the lower end part of the guide mechanism mark 18 in FIG. 4 is set as a distance H. The distance H corresponds to the distance obtained by enlarging or reducing the h in FIG. 3 by L2/L1 times. In addition, the X coordinate at the center of the lower end part of the guide mechanism mark 18 is set as X1. The “lower end” is the end part at the subject contact surface 20 side in the guide mechanism 11 when transmitting and receiving the ultrasound waves with the ultrasound probe 1. The X1 is the coordinate obtained by enlarging or reducing the x1 by L2/L1 times. Such setting of the coordinates is merely an example, and any setting which can reflect precisely the real position of the guide mechanism 11 may be used.

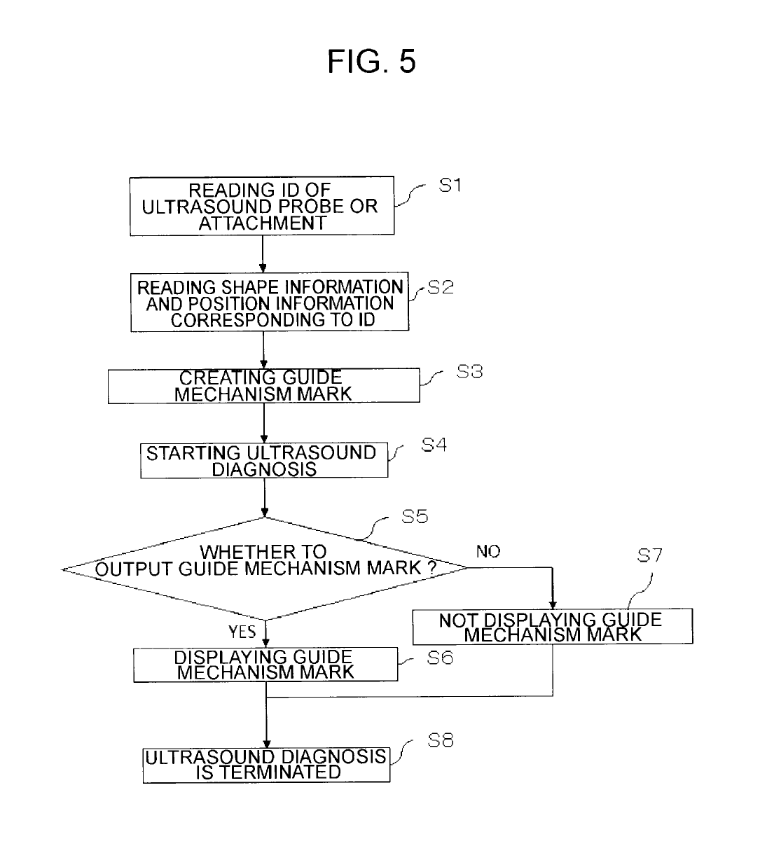

FIG. 5 illustrates the flow of the embodiment.

At S1, the guide mechanism mark setting unit 6 reads the ID of either the ultrasound probe 1 or the attachment to be used. The timing of reading the ID is at mounting of the probe 1 or the attachment, or at an arbitrary timing of the operator. If the ID is read at an arbitrary timing of the operator, the operator conducts an instruction operation to read the ID of either the ultrasound probe 1 or the attachment by the guide mechanism mark setting unit 6 via the operation unit 8.

At S2, the guide mechanism mark setting unit 6 reads the shape information and the position information corresponding to the ID read by the guide mechanism mark setting unit 6 itself out from the guide mechanism database 5.

At S3, the guide mechanism mark setting unit 6 creates the guide mechanism mark 18 based on the shape information read out from the guide mechanism database 5.

At S4, the operator starts the ultrasound diagnosis.

At S5, the operator selects whether to output the guide mechanism mark 18 on the display 7 or not.

When the operator selects to output the guide mechanism mark 18 on the display 7, the guide mechanism mark setting unit 6 transmits the guide mechanism mark 18 and the position information to the display 7. The display 7 displays the guide mechanism mark 18 transmitted from the guide mechanism mark setting unit 6 at the display position which has been set based on the position information (S6).

On the other hand, when the operator selects not to output the guide mechanism mark 18 on the display 7, the guide mechanism mark setting unit 6 does not transmit the data and the position information of the guide mechanism mark 18 to the display 7. The display 7 therefore displays only the ultrasound image 19 (S7).

At S8, the ultrasound diagnosis is terminated.

The operator pierces the puncture needle 22 at an arbitrary timing during S4 to S8.

When piercing the puncture needle 22, the operator pierces the puncture needle 22 along the wall unit 31 of the guide mechanism 11. Therefore, grasping the inclination of the wall unit 31 with respect to the subject contact surface 20 by the operator largely affects the accuracy of piercing angle and piercing position. In this respect, in the embodiment, the guide mechanism mark 18 imitating the shape of the guide mechanism 11 is caused to be displayed on the display 7. As a result, the operator can easily recognize the inclination of the wall unit 31.

Consequently, it becomes possible for the operator to recognize the direction of piercing the puncture needle 22 without impairing the visibility of the ultrasound image 19, and thus, it becomes possible to perform the ultrasound paracentesis technique smoothly.

In addition, it may be configured so that the display/non-display for the guide mechanism mark 18 is switched based on the arbitrary operation by the operator.

As a modified example of the embodiment, in addition to displaying the guide mechanism mark 18 by the not shown display controller, the limit of the piercing angle based on the shape of the guide mechanism mark 18 may be displayed on the ultrasound image 19.

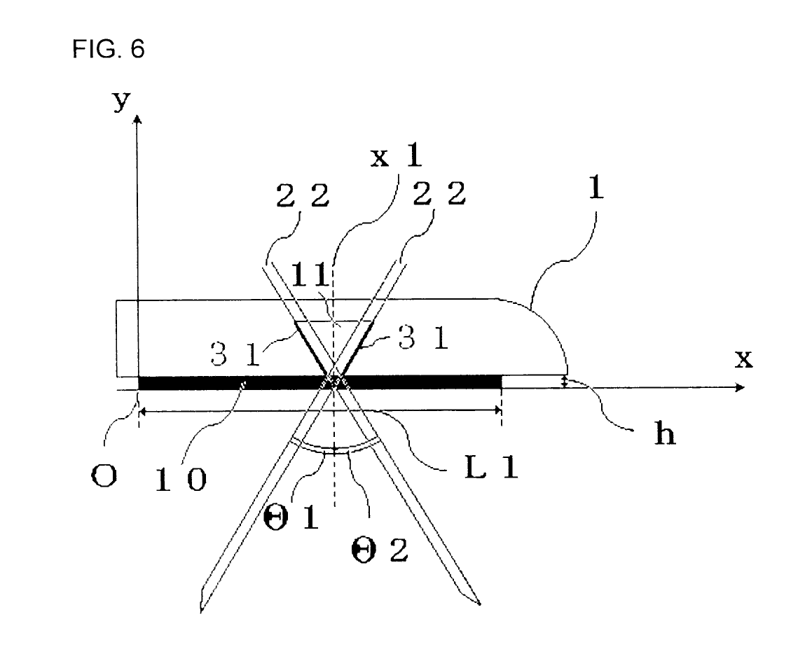

FIG. 6 is a side view of the ultrasound probe 1 when the operator pierces the puncture needle 22 along the wall unit 31 of the guide mechanism 11 and parallel to the wall unit 31. As to the coordinates in FIG. 6, the origin O corresponds to the end part of the ultrasound wave transceiver 10, as in FIG. 3. Further, the x-axis corresponds to the longitudinal direction of the ultrasound wave transceiver 10. Furthermore, the y-axis corresponds to the vertical direction to the subject contact surface 20. In the following, it will be described as the x-axis is being positioned along the subject contact surface 20. In addition, the length of the ultrasound wave transceiver 10 in the longitudinal direction is set as L1, and the distance between the x-axis and the lower end of the guide mechanism 11 is set as h. In FIG. 6, it is assumed that one kind of the guide mechanism 11 is provided, and the coordinate at the center of the lower end part of the guide mechanism 11 is set as x1.

Here, the wall unit 31 of the guide mechanism 11 is inclined with respect to a straight line which passes through the x1 and parallel to the y-axis by θ1 in the right direction and by θ2 in the left direction of the drawing. Therefore, if the operator pierces the puncture needle 22 along the wall unit 31 and parallel to the wall unit 31, as in FIG. 6, the puncture needle 22 is to be inclined with respect to the straight line which passes through the x1 and parallel to the y-axis by θ1 in the right direction and by θ2 in the left direction of the drawing.

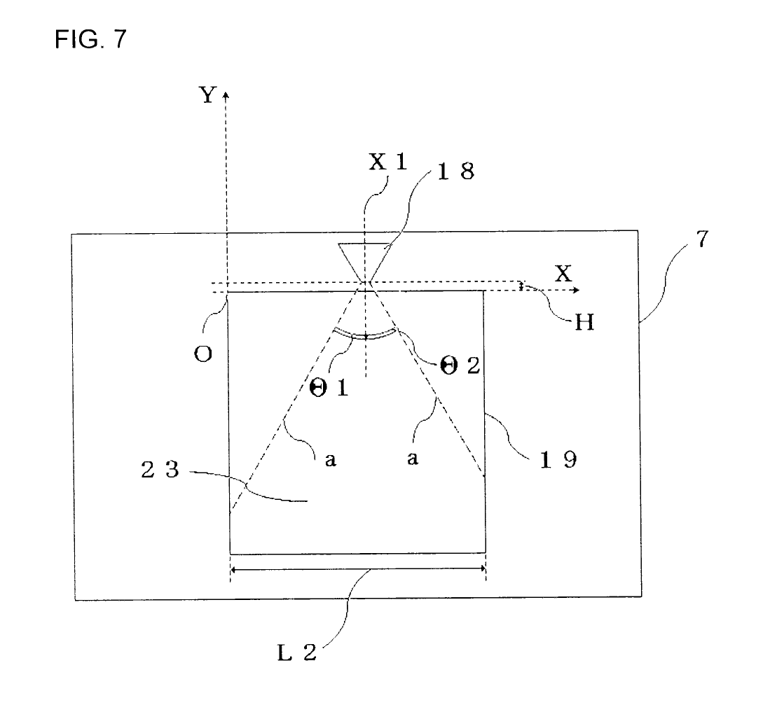

FIG. 7 is, similar to FIG. 4, an example of the screen representing the arrangement of the guide mechanism mark 18 and the ultrasound image 19 displayed on the display 7. This screen may be configured to be caused to display by a not shown display controller. The origin O in FIG. 7 corresponds to the origin O in FIG. 6. The range L2 of the ultrasound diagnosis corresponds to the range obtained by enlarging or reducing the length L1 of the ultrasound wave transceiver 10 in the longitudinal direction at a certain magnification (L2/L1 times). The distance between the upper boundary (X-axis) of the ultrasound image 19 and the lower end part of the guide mechanism mark 18 in FIG. 6 is set as the distance H. The distance H corresponds to the distance obtained by enlarging or reducing the h in FIG. 6 by L2/L1 times. The X coordinate at the center of the lower end part of the guide mechanism mark 18 is set as X1. The X1 is the coordinate obtained by enlarging or reducing the x1 by L2/L1 times. Such setting of the coordinates is merely an example, and any setting which can reflect precisely the real position of the guide mechanism 11 may be used. The display controller corresponds to an example of an “area suggestion unit” or a “boundary line creator”.

On the other hand, unlike the ultrasound image 19 in FIG. 4, the ultrasound image 19 in FIG. 7 has two dashed lines as. These two dashed lines as are inclined by θ1 and θ2 with respect to a straight line which passes through the X1 and is parallel to the Y-axis, respectively, and each corresponds to the puncture needle 22 in FIG. 6. The range sandwiched between the two dashed lines a and a is the range the image indicating the puncture needle 22 can appear in the ultrasound image 19. In the following, this range is described as a puncture needle piercing range 23. The puncture needle piercing range 23 corresponds to an example of an “area”.

For example, by the display controller, a control to show the puncture needle piercing range 23 with such two dashed lines a and a in the ultrasound image is performed. As a result, the range where the image of the puncture needle 22 appears in the ultrasound image 19 can be determined at a glance without impairing the visibility of the puncture target part 21 in the ultrasound image 19. Therefore, according to the embodiment, it is possible to simplify diagnosis and reduce diagnosis time.

Further, as with the guide mechanism mark 18, it may be configured to switch the display/non-display for the puncture needle piercing range 23 arbitrarily by the operator. Furthermore, it may be configured to display by changing colors in the puncture needle piercing part 23 and in the other parts. Such configuration makes it possible to emphasize the puncture needle piercing range 23 without impairing the visibility of the puncture needle piercing range 23.

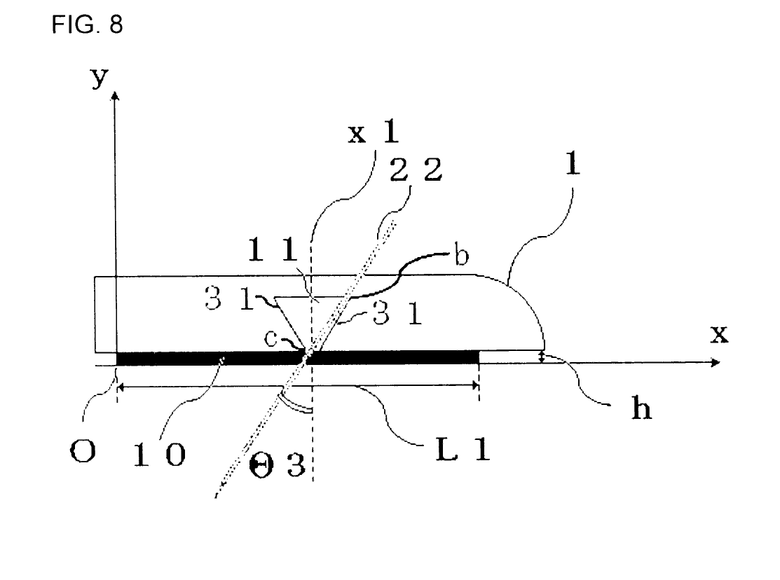

In addition, if the width of the puncture needle 22 is smaller than the width of the lower end part of the guide mechanism 11, as in FIG. 8, the inclination of the puncture needle 22 becomes maximum (θ3) in the state where the puncture needle 22 is contacted with both the upper end b of one part of the wall unit 31 and the lower end c of the other part of the wall unit 31. In this case, the puncture needle piercing range 23 may reflect the θ3.

In the embodiment, a member which detects the passing of the puncture needle 22 may be provided to the wall unit 31, or the like, of the guide mechanism 11 and the like. This member is, for example, a photo censor. In this case, the photo censor detects the puncture needle 22 passes the guide mechanism 11. As a result of the detection, the guide mark mechanism mark 18 and the puncture needle piercing range 23 are automatically displayed. The member is not limited to the photo censor and any member which can detect the passing of the puncture needle 22 may be used.

### Second Embodiment

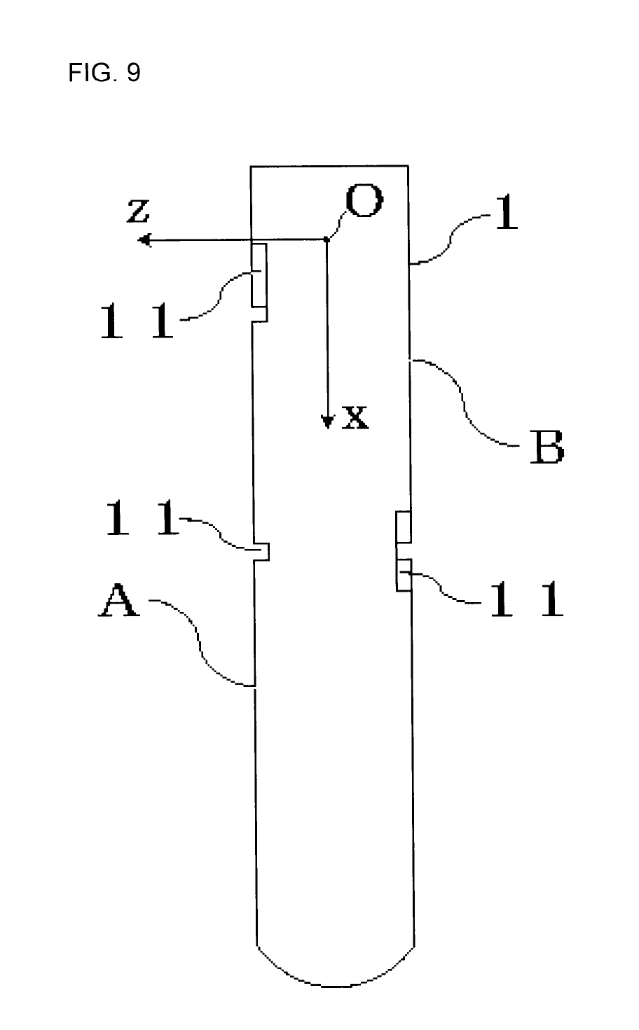

In a second embodiment, as shown in FIG. 9, the case in which the guide mechanisms 11 is provided on the different side surfaces of the ultrasound probe 1 is described. Further, the case in which the ultrasound probe 1 has a plurality of guide mechanisms 11 on one side surface is described. In addition, as described above, there is a case in which instead of the ultrasound probe 1, a not shown attachment is used. In this case, the “ultrasound probe 1” in the following description is read as an “attachment”.

FIG. 9 is a plan view of the ultrasound probe 1 in the embodiment. The side surface at left side is set as A-side, and the side surface at right side is set as B-side in FIG. 9. The A-side and B-side in FIG. 9 are side surfaces along the longitudinal direction of the ultrasound probe 1. Also, the B-side is the side surface positioned opposite side of the A-side.

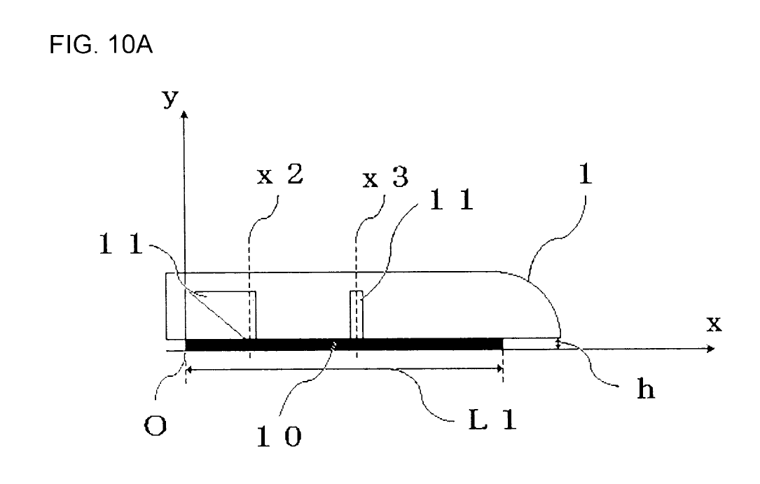

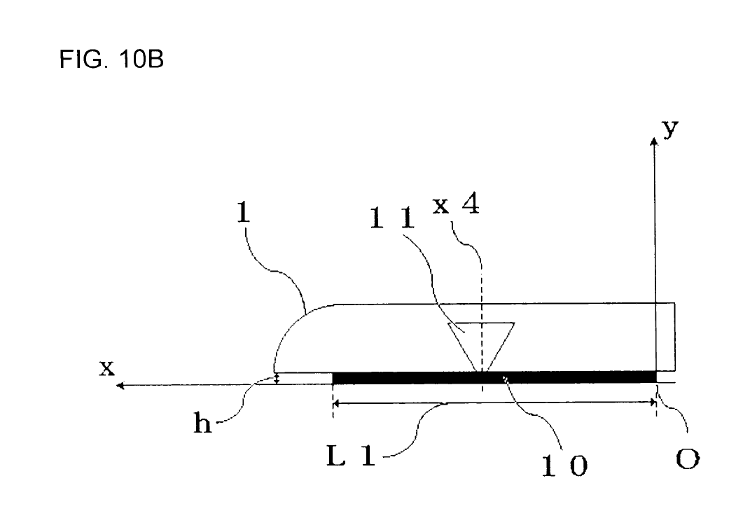

FIG. 10A shows the ultrasound probe 1 at the A-side. FIG. 10B shows the ultrasound probe 1 at the B-side. In addition, the coordinates in FIG. 10A correspond to those in FIG. 10B.

In the second embodiment, the coordinates are also set in a similar manner to FIG. 3.

The origin O corresponds to the end part of the ultrasound wave transceiver 10. Further, the x-axis corresponds to the longitudinal direction of the ultrasound wave transceiver 10. Furthermore, the y-axis corresponds to the vertical direction to the subject contact surface 20. Again, it is assumed that the x-axis is positioned along the subject contact surface 20. In addition, the length of the ultrasound wave transceiver 10 in the longitudinal direction is set as L1, and the distance between the X-axis and the lower end of the guide mechanism 11 is set as h. The “lower end” is the end part at the subject contact surface 20 side in the guide mechanism 11 when transmitting and receiving ultrasound waves with the ultrasound probe 1.

The coordinates at the center of the lower end part of the guide mechanism 11 in FIG. 10A are set as x2 and x3 in sequence from the left side in FIG. 10A. Further, the coordinate at the center of the lower end part of the guide mechanism 11 in FIG. 10B is set as x4.

The operator selects either the guide mechanism mark 18 at the A-side or the guide mechanism mark 18 at the B-side is to be displayed via the operation unit 8 and the system controller 9. For example, a switch and the like may be provided to the operation unit 8, or the options may be displayed on the screen displayed on the display 7. The options may be configured to be caused to display by a not shown display controller.

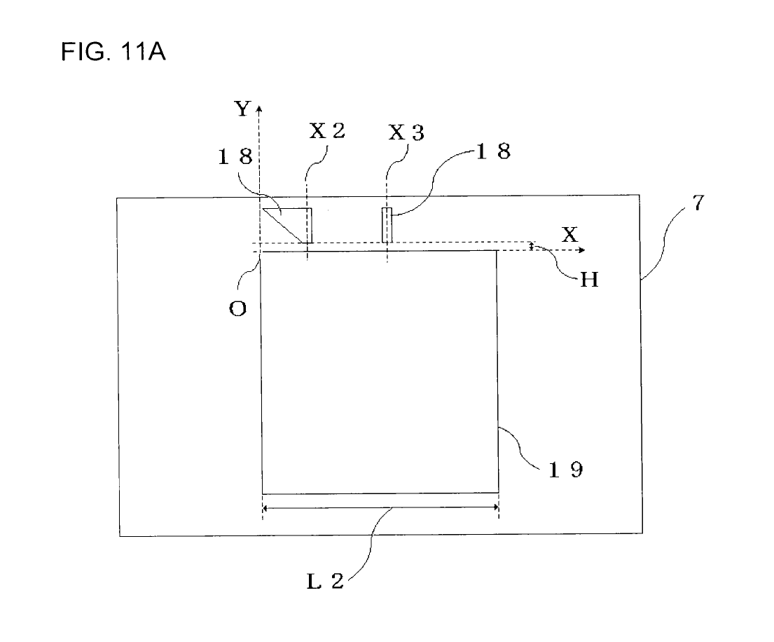

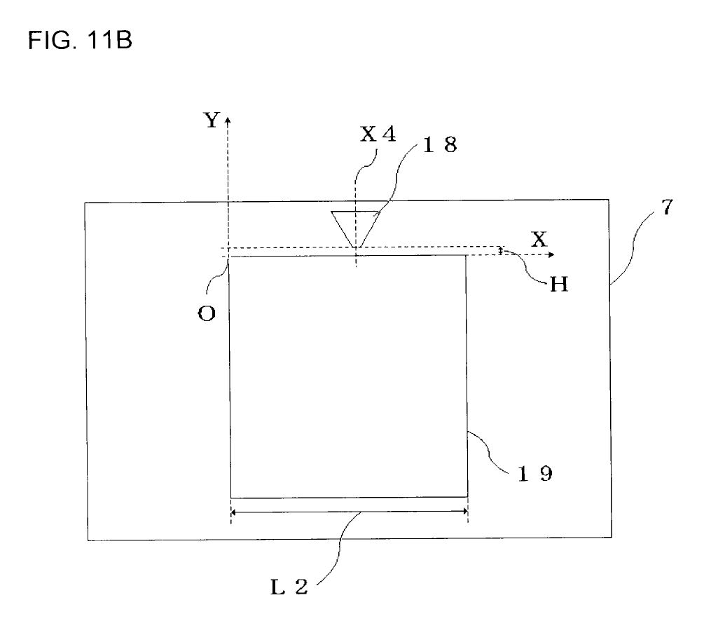

The FIG. 11A and FIG. 11B are examples of a screen representing the ultrasound image 19 and the guide mechanism mark 18 displayed on the display 7. The screen may be configured to be caused to display by a not shown display controller.

If the operator selects to display the guide mechanism mark 18 at the A-side, FIG. 11A corresponding to FIG. 10A is displayed on the display 7. The origin O in FIG. 11A corresponds to the origin O in FIG. 10A. The range L2 of the ultrasound diagnosis corresponds to the range obtained by enlarging or reducing the length L1 of the ultrasound wave transceiver 10 in the longitudinal direction at a certain magnification (L2/L1 times). The distance between the upper boundary (X-axis) of the ultrasound image 19 and the lower end part of the guide mechanism mark 18 in FIG. 11A is set as H. The H corresponds to the distance obtained by enlarging or reducing the h in FIG. 10A by L2/L1 times.

The X coordinates at the center of the lower end part of the guide mechanism mark 18 in FIG. 11A are set as X2 and X3 in sequence from the left side in FIG. 11A. The X2 and X3 are the coordinates obtained by enlarging or reducing the x2 and x3 by L2/L1 times, respectively.

On the other hand, if the operator selects to display the guide mechanism mark 18 at the B-side, FIG. 11B corresponding to FIG. 10B is displayed on the display 7. The origin O in FIG. 11B corresponds to the origin O in FIG. 10B. The range L2 of the ultrasound diagnosis is the range obtained by enlarging or reducing the L1 of the ultrasound wave transceiver 10 in the longitudinal direction at a certain magnification (L2/L1 times). The distance between the upper boundary (X-axis) of the ultrasound image 19 and the lower end part of the guide mechanism mark 18 in FIG. 11B is set as H. The distance H corresponds to the distance obtained by enlarging or reducing the h in FIG. 10B by L2/L1 times.

The X coordinate at the center part of the lower end part of the guide mechanism mark 18 in FIG. 11B is set as X4. The X4 is the coordinate obtained by enlarging or reducing the x4 by L2/L1 times.

In addition, it may be configured so that the guide mechanism marks 18 at both A-side and B-side are displayed at the same time without changing displays for each side surface as described above. In this case, the colors may be changed in the guide mechanism mark 18 at the A-side and in the guide mechanism mark 18 at the B-side.

Further, in the second embodiment, it may be configured to display the puncture needle piercing range 23 as in the first embodiment.

Consequently, the operator can recognize the direction for piercing the puncture needle 22 without impairing the visibility of the ultrasound image 19, and thereby the smooth ultrasound paracentesis technique can be performed.

Additionally, as a modified example, it may be configured to detect the inclination of the ultrasound probe 1 by providing a jayro censor, or the like, to the ultrasound probe 1. For example, when the A-side in FIG. 10 faces upward, the guide mechanism mark 18 at the A-side or the puncture needle piercing range 23 is automatically displayed on the display 7.

Further, when the operator pierces the puncture needle 22 in a state where the ultrasound probe 1 is inclined, the lower end of the guide mechanism 11 is sometimes contacted with the subject contact surface 20. In this case, the h in FIG. 3 becomes substantially 0, and the H in FIG. 5 also becomes 0 accordingly. Further, although, it is described assuming that the ultrasound image 19 corresponding to the whole ultrasound wave transceiver 10 is displayed in the embodiment, the embodiment can be applied to a case where the ultrasound image 19 corresponding to a part of the ultrasound transceiver 10 is displayed. That is, in such a case, it is also possible to display the guide mechanism mark 18 in accordance with the real position of the guide mechanism 11.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed:

1. An ultrasound diagnosis apparatus, comprising:
an ultrasound probe configured to transmit/receive ultrasound waves;
a guide mechanism which is provided directly or indirectly to the ultrasound probe and has a wall unit configured to regulate so as to allow an angle with which a puncture needle is guided to a puncture target part within a predetermined range;
an image processor configured to generate an ultrasound image from echo signal received by the ultrasound probe;
a display configured to display the ultrasound image; and
a marker output unit configured to output a marker indicating the inclination of the wall unit on the display.

2. The ultrasound diagnosis apparatus according to claim 1, wherein the marker output unit is configured to identify an output position of the marker corresponding to the position of the guide mechanism from the position relationship between the ultrasound probe and the ultrasound image so as to output the marker at the identified output position.

3. The ultrasound diagnosis apparatus according to claim 2, comprising:
a storage configured to store guide mechanism information including the shape and the position of the guide mechanism;
a selection unit configured to select the guide mechanism information corresponding to the guide mechanism for use from the guide mechanism information stored in the storage; and
a marker creator configured to create the marker based on the guide mechanism information selected by the selection unit.

4. An ultrasound diagnosis apparatus, comprising:
an ultrasound probe configured to transmit/receive ultrasound waves;
a guide mechanism which is provided directly or indirectly to the ultrasound probe and is configured to guide an puncture needle to a puncture target part;
an image processor configured to generate an ultrasound image from echo signals received by the ultrasound probe;
a display configured to display the ultrasound image; and
an area suggestion unit configured to suggest an area in which an image of the puncture needle based on the shape of the guide mechanism can appear on the display.

5. The ultrasound diagnosis apparatus according to claim 4, wherein the area is the area sandwiched between two boundary lines indicating a limit in which the puncture needle can be pierced along the wall unit of the guide mechanism, and the area suggestion unit is configured to identify the output positions of the two boundary lines corresponding to the position of the guide mechanism from the position relationship between the ultrasound probe and the ultrasound image so as to output the two boundary lines at the output positions.

6. The ultrasound diagnosis apparatus according to claim 5, comprising:
a storage configured to store guide mechanism information including the shape and the position of the guide mechanism;
a selection unit configured to select the guide mechanism information corresponding to the guide mechanism for use from the guide mechanism information stored in the storage; and
a boundary line creator configured to create the two boundary lines based on the guide mechanism information selected by the selection unit.

7. An ultrasound diagnosis apparatus, comprising:
an ultrasound probe configured to transmit/receive ultrasound waves;
a guide mechanism which is provided directly or indirectly to the ultrasound probe and is configured to guide a puncture needle to a puncture target part;
an image processor configured to generate an ultrasound image from echo signals received by the ultrasound probe;
a display configured to display the ultrasound image;
a marker output unit configured to output a marker imitating the shape for the guide mechanism on the display; and
an area suggestion unit configured to suggest an area in which an image of the puncture needle based on the shape of the guide mechanism can appear on the display.

8. An ultrasound diagnosis apparatus, comprising:
an image processor configured to generate an ultrasound image based on echo signals received by an ultrasound probe; and
a display controller configured to cause a display unit to display, along with the ultrasound image, a marker indicating an allowable change range for an orientation of a puncture needle defined by a guide mechanism for guiding the puncture needle to a puncture target part.

9. The ultrasound diagnosis apparatus according to claim 8, wherein the marker is configured to indicate the allowable change range defined by the shape of the guide mechanism.

10. The ultrasound diagnosis apparatus according to claim 8, wherein the marker is configured to indicate the allowable change range defined by the shape of the wall unit of the guide mechanism.

11. The ultrasound diagnosis apparatus according to claim 8, wherein the display controller is configured to cause the marker to be displayed at a position adjacent to the ultrasound image.

12. The ultrasound diagnosis apparatus according to claim 8, wherein the display controller is configured to cause the marker to be displayed based on the positions of the ultrasound probe and the guide mechanism.

13. The ultrasound diagnosis apparatus according to claim 1, wherein the image processor is configured to generate an ultrasound image from echo signals received by the ultrasound probe in a state where at least most of the guide mechanism is inserted into a subject.

14. The ultrasound diagnosis apparatus according to claim 11, wherein the display controller is configured to cause the marker to be displayed based on the positions of the ultrasound probe and the guide mechanism.

15. The ultrasound diagnosis apparatus according to claim 4, wherein the image processor is configured to generate an ultrasound image from echo signals received by the ultrasound probe in a state where at least most of the guide mechanism is inserted into a subject.

16. The ultrasound diagnosis apparatus according to claim 7, wherein the image processor is configured to generate an ultrasound image from echo signals received by the ultrasound probe in a state where at least most of the guide mechanism is inserted into a subject.

17. The ultrasound diagnosis apparatus according to claim 8, wherein the image processor is configured to generate an ultrasound image from echo signals received by the ultrasound probe in a state where at least most of the guide mechanism is inserted into a subject.
